(19) United States
(12) Patent Application Publication
(10) Pub. No.: US 2009/0017006 A1
(43) Pub. Date: Jan. 15, 2009

(54) USE OF A COMPOUND FOR ENHANCING THE EXPRESSION OF MEMBRANE PROTEINS ON THE CELL SURFACE

(75) Inventors: Michael Freissmuth, Vienna (AT); Tetyana Milojevic, Vienna (AT); Christian Nanoff, Vienna (AT); Volodymyr M. Korkhov, Cambridge (GB)

Correspondence Address: FITZPATRICK CELLA HARPER & SCINTO, 30 ROCKEFELLER PLAZA, NEW YORK, NY, 10112, US

(73) Assignee: BIODEVELOPS PHARMA ENTWICKLUNG GMBH, Wien (AT)

(21) Appl. No.: 11/994,947

(22) PCT Filed: Jul. 3, 2006
(86) PCT No.: PCT/AT06/00282

§ 371 Date: Jul. 28, 2008

(30) Foreign Application Priority Data
Jul. 6, 2005 (AT) 1144/2005

(51) Int. Cl. A61K 38/48; A61K 31/4965; A61K 31/7088; C12P 21/04; C12P 21/06

(52) U.S. Cl. 424/94.63; 514/255.06; 514/44; 435/70.1; 435/68.1; 435/69.1

(57) ABSTRACT

The present invention is directed to the use of Bortezomib and/or a pharmaceutically acceptable salt or ester thereof for the manufacture of a medicament for enhancing the expression of membrane proteins on the cell surface. Especially, the invention is directed to the use of Bortezomib for the manufacture of a medicament for the treatment of a disease of condition selected from the group consisting of cystic fibrosis, diabetes insipidus, hypercholesterinaemia and long QT-syndrome-2.

### BACKGROUND OF THE INVENTION

1. Field of the Invention

Membrane proteins, especially integral membrane proteins, have to be inserted cotranslationally into the endoplasmic reticulum. This occurs via the translocon, which is a channel formed by the Sec61-subunits. During and after synthesis of membrane proteins in the endoplasmic reticulum, they undergo a strict quality control to ensure correct folding before they are transported to their definitive site of action.

2. Prior Art

Several aspects of this quality control are incompletely understood; nevertheless it is clear that incorrectly folding of a membrane protein is sensed by the machinery of the endoplasmic reticulum (that is by chaperons, presumably). This leads to activation of ubiquitinating enzymes on the cytoplasmic side. These transfer ubiquitin to the cytoplasmic peptide chain of the incorrectly folded protein which is retrotranslocated through the Sec61 channel and degraded by the 26S proteasome (Kostova and Wolf, 2003). It has to be stressed that this scheme relies predominantly on observations that were made in Saccharomyces cerevisiae. Based on several pieces of experimental evidence, it is, however, reasonable to assume that the higher eukaryotes employ a related machinery to eliminate misfolded proteins.

It has been increasingly appreciated that many human diseases can be linked to mutations, which result in the retention of the aberrant protein in the endoplasmic reticulum (ER). Cystic fibrosis is most commonly cited as the model disease: More than 1000 mutations have been identified in the gene encoding the CFTR (cystic fibrosis transmembrane conductance regulator) (Rowntree and Harris, 2003), but the majority of the patients (˜70%) have the ΔF508-mutation of the CFTR.

The resulting protein can function properly, if it reaches the plasma membrane; however, it fails to reach the plasma membrane due to an overprotective ER quality control mechanism (Pasyk and Foskett, 1995). There are many more examples that lead to defective ER-export of membrane proteins; these include mutations of the V₂-vasopressin receptor (associated with diabetes insipidus; Oksche and Rosenthal, 1998), of the LDL-receptor (resulting in hypercholesterinaemia; Hobbs et al., 1990; Jorgensen et al., 2000), or of the HERG-K⁺-channel (resulting in long QT-syndrome-2; Kupershmidt et al., 2002) etc.

It is unclear why these mutated proteins are retained and eventually degraded although they are—at least in part—functionally active (see Pasyk and Foskett, 1995). However, the available evidence suggests that the quality control machinery in the endoplasmic reticulum is overprotective.

It is known that proteasome inhibitors may enhance the expression of membrane proteins on the cell surface, cf. e.g. Jensen T J et al.; Cell. 1995 Oct. 6; 83(1): 129-35.

Furthermore, it has been found (U.S. patent application Ser. No. 10/886,202, unpublished) that de-ubiquitinating enzymes, such as USP-4 are useful in enhancing the expression of membrane proteins on the cell surface.

### SUMMARY OF THE INVENTION

It is an object of the present invention to provide means for enhancing the expression of membrane proteins, especially integral membrane proteins, on the cell surface. Especially, it is an object of the present invention to provide means for enhancing the expression of a protein selected from the group consisting of CFTR (cystic fibrosis transmembrane conductance regulator), V₂-vasopressin receptor, LDL-receptor and HERG-K⁺-channel and, furthermore, to provide a medicament for the treatment of a disease or condition selected from the group consisting of cystic fibrosis, diabetes insipidus, hypercholesterinaemia and long QT-syndrome-2.

This object is achieved by the subject matter of the independent claims. Preferred embodiments are disclosed in the dependent claims.

Bortezomib (N-(2-pyrazine)carbonyl-L-phenylalanine-L-leucine-boronic acid) is a known anti-cancer agent with proteasome-inhibiting activity (EP 0 788 360 A, EP 1 123 412 A, WO 04/156854).

While proteasome inhibitors such as MG132 have been found to cause cell apoptosis even at very small administration dosage, it has surprisingly been found that there is a therapeutic window for administering Bortezomib, whereby expression of membrane proteins such as CFTR or its most common ΔF508-mutation is enhanced whilst no increased cell mortality is observed.

In the case of HE 293 cells, this therapeutical window is between 1 nM and 100 nM Bortezomib, preferably from 3 nM to 10 nM. The skilled artisan can easily adapt the pharmaceutically acceptable dosis of Bortezomib depending on the disease to be treated.

In addition, stimulating the deubiquitinating activity in a cell, especially by increasing the amount of deubiquitinating enzymes in the cell or stimulating them, furthermore enhances the expression of integral membrane proteins on the cell surface. Especially, deubiquitinating enzymes are capable of decreasing the level of overprotective quality control in the endoplasmatic reticulum.

Increasing the amount of deubiquitinating enzymes in the cell can be achieved especially by introducing into the cell a compound selected from the group consisting of

a deubiquitinating enzyme

a nucleic acid sequence encoding a deubiquitinating enzyme.

Especially, the cell may be transfected with an appropriate plasmid containing DNA encoding the deubiquitinating enzyme, followed by expression of the enzyme in the cell.

The ways to introduce a deubiquitinating enzyme or the nucleic acid sequence encoding the enzyme, as well as identifying suitable amounts of compound to be introduced, are known to the skilled artisan or can be determined using knowledge which is well available to the skilled artisan.

Preferably the deubiquitinating enzyme is selected from the group consisting of ubiquitin carboxy-terminal hydrolases (UCH) and ubiquitin specific proteases (USP). USPs are also being referred to as ubiquitin processing proteases (UBPs; Wing, 2003).

Deubiquitinating enzymes are thiol proteases which hydrolyse the amide bond between Gly76 of ubiquitin and the substrate protein. There are two classes of deubiquitinating enzymes; the ubiquitin-specific processing protease or USP class is one of these two known classes of deubiquitinating enzymes (Papa and Hochstrasser, 1993). While the catalytic activity has been tested using artificial substrates, very little is known about their physiological substrates and thus their physiological functions. USPs have been shown to play a role in determination of cell fate (fat facets; Huang et al. (1995), transcriptional silencing (UBP3; Moazed and Johnson, D. (1996)), response to cytokines (DUB1 and 2; Zhu et al., 1996) and oncogenic transformation (tre-2, USP4; Gilchrist and Baker, 2000), but the mechanistic details have remained enigmatic.

In an especially preferred embodiment, the deubiquitinating enzyme is USP-4. The sequence of murine USP-4 enzyme is, for example, disclosed in Strausberg, R. L., et al.; Proc. Natl. Acad. Sci. U.S.A. 99 (26), 16899-16903 (2002). Human USP-4 exists in two variants, cf. Puente, X. S. et al., Nat. Rev. Genet. 4 (7), 544-558 (2003).

The method of the present invention enables especially expression of a protein selected from the group consisting of CFTR (cystic fibrosis transmembrane conductance regulator), V₂-vasopressin receptor, LDL-receptor and HERG-K⁺-channel.

Furthermore, the method of the present invention can be used for the treatment of conditions or diseases related to or associated with the lack of expression of membrane proteins on the cell surface.

Especially, the method of the present invention enables treatment of a disease or condition selected from the group consisting of cystic fibrosis, diabetes insipidus, hypercholesterinaemia and long QT-syndrome-2.

The present invention is also directed to a pharmaceutical composition, comprising a therapeutically effective amount of Bortezomib and/or a pharmaceutically acceptable salt or ester thereof, and a compound stimulating deubiquitinating activity in a cell.

Preferably, said compound is selected from the group consisting of

a deubiquitinating enzyme

a nucleic acid sequence encoding a deubiquitinating enzyme.

### BRIEF DESCRIPTION OF THE FIGURES

FIG. 1 shows immunoblots of membranes from cells transfected with GFP-tagged CFTR and CFTR-Δ508, respectively, and having undergone different treatments.

FIGS. 2a, 2b and 2c, respectively, show the result of fluorescence activated cell sorting (FACS)-monitoring of the expression of GFP-tagged CFTR from HEK293 cells.

FIGS. 3a, 3b and 3c, respectively, show the result of FACS-monitoring of the expression of GFP-tagged CFTR-Δ508 from HEK293 cells.

FIG. 4 shows the effect of 10 nM Bortezomib on the expression of GFP-tagged CFTR-Δ508 from HEK293 cells.

FIG. 5 shows the effect of 100 nM Bortezomib on the expression of GFP-tagged CFTR-Δ508 from HEK293 cells.

FIG. 6 shows the effect of 1 Mm Bortezomib on the expression of GFP-tagged CFTR-Δ508 from HE 293 cells.

FIG. 7 shows the effect of 1 Mm MG 132 on the expression of GFP-tagged CFTR-Δ508 from HE 293 cells.

FIGS. 8 and 9 show the comparison of expression of GFP-tagged CFTR-Δ508 from HEK293 cells which have not been co-transfected with USP-4 (FIG. 8) and cells which have been co-transfected with USP-4 (FIG. 9).

### DETAILED DESCRIPTION OF THE INVENTION

### Examples

In the following examples, the effect of USP-4, MG 132 and Bortezomib, respectively, on the expression of the ΔF508-mutation of CFTR was examined.

### Materials and Methods

### Immunoblot for CFTR and CFTR-ΔF508 Expressed in HEK293 Cells:

HEK293 cells (1*10⁶ cells) were transfected with plasmids encoding CFTR or CFTR-ΔF508 (GFP-tagged) and/or co-transfected with effector plasmids. After 16 h, the cells were treated with the varying concentrations of compounds. After 24 h, the cells were harvested in phosphate-buffered saline, lysed by a freeze-thaw cycle and homogenized by sonication. The homogenate was resuspended in reducing Laemmli sample buffer (50 mM Tris.Hcl, pH 6.8, 20% glycerol, 0.1% bromophenol blue, 2% SDS and 20 mM dithiothreitol); aliquots (15% of the original culture) were resolved on a denaturing polyacrylamide gel (monomer concentration in the stacking gel and in the running gel 4 and 8% respectively) and electrophoretically transferred to a nitrocellulose membrane. Immunodetection was done with an antiserum directed against GFP as the primary antibody and an anti-rabbit IgG coupled to horseradish peroxidase as the secondary antibody. Immunoreactive bands were revealed by enhanced chemiluminescence (ECL kit, Super Signal Pierce).

### Fluorescence Activated Cell Sorting (FACS)

Cultured HEK293 cells were transfected with plasmids encoding CFTR or CFTR-ΔF508 (GFP-tagged) and/or co-transfected with plasmids encoding USP4 (or an appropriate control plasmid) by using the CaPO₄-precipitation method. Sixteen hours after transfection the cells were treated with varying concentrations of compounds. At a specific time point (here 24 h) the cells are trypsinized, fixed in ethanol, permeabilized and stained with propidium iodide (PI). The stained cells are subjected to FACS analysis

### Results

### USP-4, MG 132 and Bortezomib Enhance Expression of the CFTR-ΔF508 Mutation:

In a first example, Membranes from transfected cells were prepared and immunoblotted for GFP-tagged CFTR or CFTR-ΔF508, respectively (by using an antibody directed against the fluorescent protein).

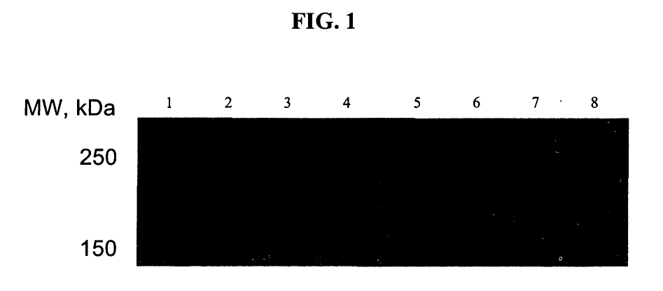

FIG. 1 shows that CFTR accumulates as a protein of ˜170 kDa, i.e. the size expected for the sum of the mass CFTR and GFP (FIG. 1, 2nd lane).

The membrane extract was also treated endoglycosidase H. The rationale for this experiment is as follows: membrane proteins are core glycosylated in the endoplasmatic reticulum. Core glycosylation is sensitive to endoglycosidase H. If the protein has reached the Golgi (and then trafficked to the plasma membrane), it acquires additional sugar moieties and becomes resistant to endoglycosidase H. It is evident from lane 3 in FIG. 1 that endoglycosidase H treatment reduces the apparent size of CFTR; thus, the bulk of the protein is still in the ER. The following lanes examine the expression of CFTR-ΔF508 (all extracts were treated with endoglycosidase H): lane 4 is the control, that is cells expressing CFTR-ΔF508; in lanes 5, 6, 7 and 8 cells expressing CFTR-ΔF508 were treated overnight (i.e. for 16 h) with 100 nM MG132, 20 mM kifunensine, 1 mM and 100 nM bortezomib, respectively. If one compares the intensity of staining of these lanes to lane 4, it is evident that all treatments—with the exception of MG132—led to the accumulation of CFTR-ΔF508. It is also evident that 100 nM bortezomib (last lane on the right hand side) was more effective than 1 μM bortezomib (adjacent lane).

### Monitoring of Expression of CFTR and CFTR-ΔF508 via FACS

Because CFTR is tagged with a fluorescent protein, expression in individual cells can be monitored by fluorescence activated cell sorting (FACS). By contrast with fluorescence microscopy (where individual cells are picked), FACS allows to survey the entire cell population. In addition, FACS has the advantage that it allows for reasonable sample throughput; finally, automation and scale-up is readily possible.

Transiently transfected HE 293 cells were fixed in ethanol 24 h after transfection as mentioned above and then stained with propidium iodide to label the DNA: the rationale was to examine the distribution of cells in the cell cycle (=to see if the expression of CFTR or of CFTR-ΔF508 was toxic or if the compounds employed killed the cells/drove them into apoptosis).

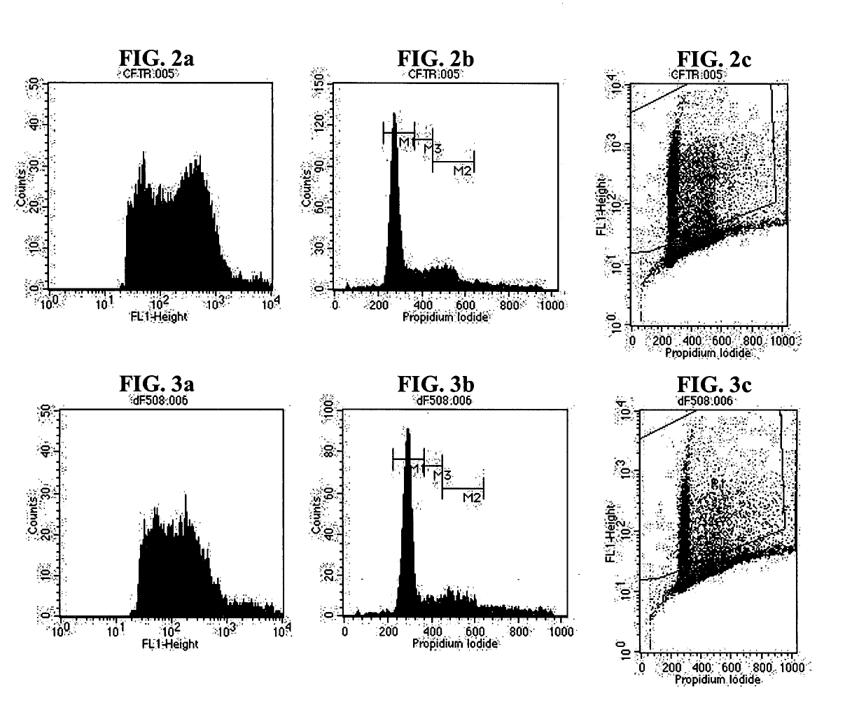

The original data set is shown on the right hand side of the figures, respectively (see e.g. FIG. 2c): the x-axis is the propidium iodide fluorescence (note that the scale is linear). The y-axis is the GFP-fluorescence (=fluorescence associated with CFTR; note that the scale is logarithmic) and each dot corresponds to a cell. The quadrangle delineates the cells that express CFTR.

One can plot the cell counts against the propidium iodide fluorescence of the transfected cells (such as shown in, for example, FIG. 2b): This gives a peak of cells (denoted by M1) that have a 2n content of DNA (G1-cells), a shoulder of cells that have a DNA content of larger than 2n (denoted by M3 and representing cells that are in S-phase) and a second peak of cells that have a DNA content of 4n (denoted by M2 and representing cells in G2 and M-phase).

The distribution of cells expressing CFTR and CFTR-ΔF508 was comparable (cf. FIG. 2, showing the result of CFTR expression and FIG. 3, showing the result of CFTR-ΔF508-expression) and comparable to that seen in untransfected cells (not shown). Thus, expression of these proteins is not toxic.

FIG. 2a and FIG. 3a, respectively, show the distribution of CFTR- or CFTR-ΔF508-associated fluorescence. It is evident that CFTR accumulates on average to higher levels: the peak is seen at 3-4*10² fluorescence units, while for CFTR-Δ508 the peak is at 102 fluorescence units.

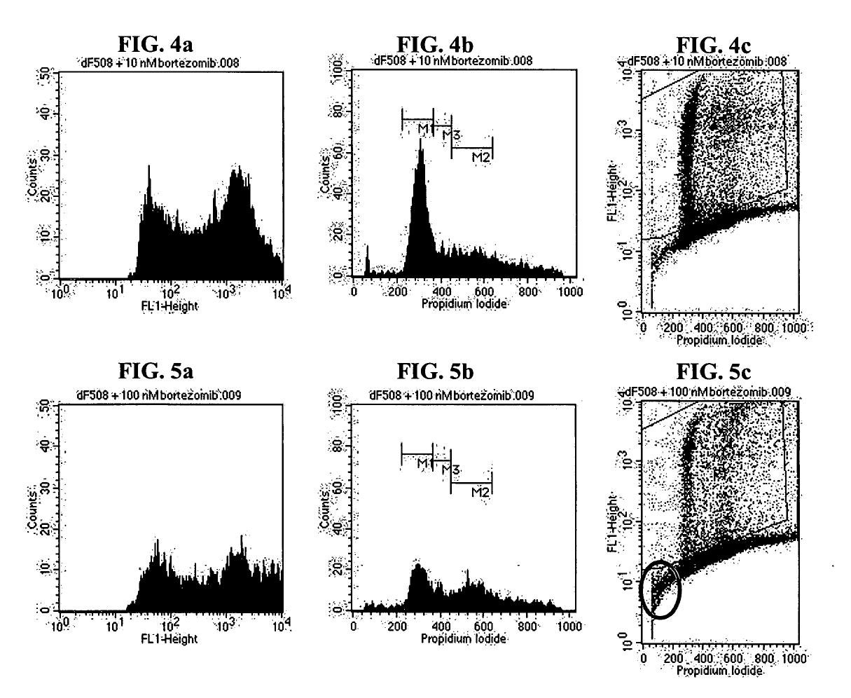

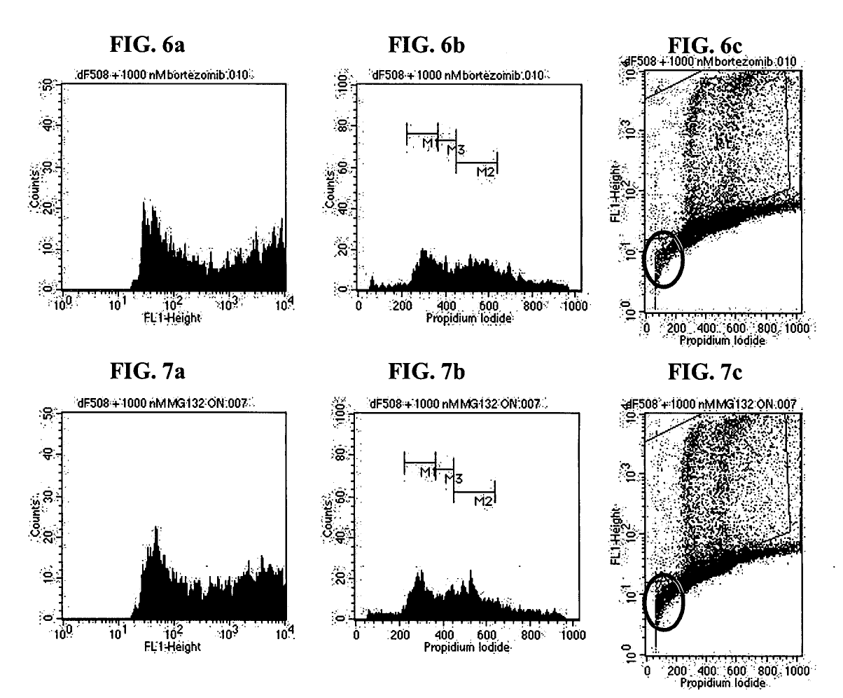

FIGS. 4, 5, 6 and 7 document the effect of increasing concentrations of bortezomib administered to the cells (10 n M —FIG. 4; 100 nM —FIG. 5; 1 μM —FIG. 6) and of 1 mM MG132 (FIG. 7, bottom) on the expression of CFTR-ΔF508. If one compares the CFTR-ΔF508-associated fluorescence in FIGS. 4a and 5a to the control (FIG. 3a), it is evident that the expression of CFTR is increased (the fluorescence shifts to higher intensities; please note again that the axis is logarithmic).

However, if one examines the original data set (FIG. 3c and FIG. 5c and FIG. 6c, respectively), it is evident that the number of cells with low propidium iodide fluorescence increases (marked by an ellipse in FIG. 5c and FIG. 6c) with increased Bortezomib concentration: these cells are apoptotic and have shut down translation (i.e. they do not make CFTR-ΔF508 and are hence not found in the quadrangle).

Thus if one examines the cell cycle distribution of CFTR-ΔF508 expressing cells (FIGS. 5b, 6b), one can see that cells in G1 are particularly sensitive to proteasome inhibition (the peak of the G1-cells—denoted by M1—is greatly reduced).

This is however not the case with 10 nM bortezomib (FIG. 4b): the cell cycle distribution is essentially the same as the one shown in control cells expressing CFTR-ΔF508 (FIG. 3b). Nevertheless, bortezomib substantially increases the level of CFTR-ΔF508 (FIG. 3c and FIG. 4c).

FIG. 7 demonstrates the effect of 1 μg MG 132 on HEK293 cells: As with Bortezomib at higher dosages, while MG 132 enhances CFTR-ΔF508-expression, there is also a pronounced apoptotic effect to be observed.

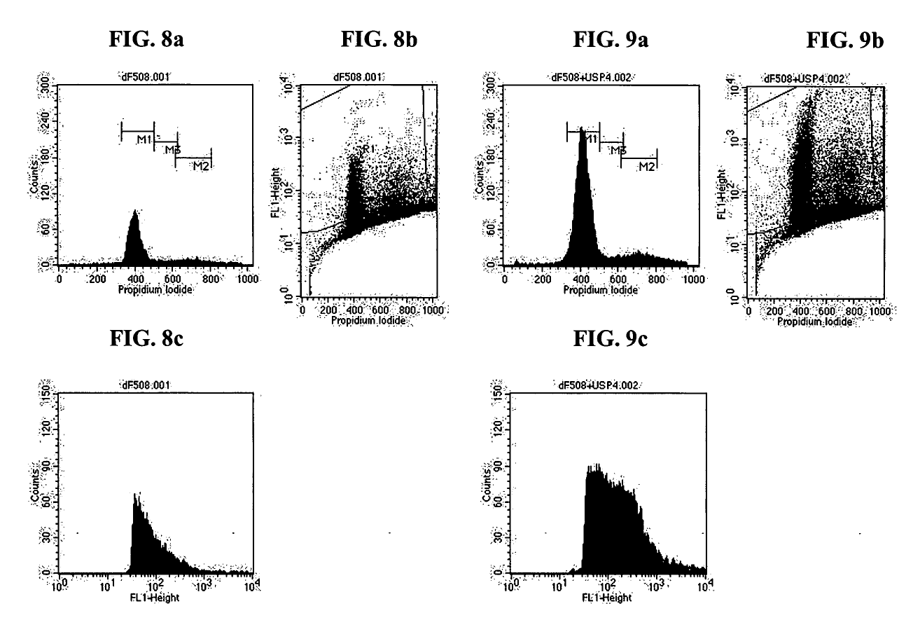

Using the FACS assay, it was furthermore tested whether enzymatic deubiquitination by USP-4 raised the accumulation of CFTR-ΔF508; this is documented in FIGS. 8 and 9, respectively: The control situation is shown in FIG. 8: i.e. the original data set with the quadrangle defining the GFP-expressing cells=CFTR-ΔF508-expressing cells (FIG. 8b), the cell cycle distribution based on the propidium iodide fluorescence (FIG. 8a) and the level of GFP-(=CFTR-ΔF508)-associated fluorescence (FIG. 8c).

FIG. 9 shows the data set for cells cotransfected with a plasmid driving the expression of USP4: A comparison of FIG. 8c and FIG. 9c readily shows that the CFTR-ΔF508-associated fluorescence increases upon co-expression of USP4 (please note again the logarithmic scale): Under control conditions (FIG. 8c), there are essentially no cells at 103 fluorescence units; in contrast, in the presence of USP-4, there is a substantial portion of cells containing CFTR-ΔF508-associated fluorescence at this range (FIG. 9c). Finally, if one compares the distribution of propidium iodide-fluorescence (FIG. 8a and FIG. 9a, respectively), it is evident that expression of USP4 does not affect the cell cycle distribution and does not increase the fraction of cells in the sub-2n fraction. In other words: expression of USP-4 is not toxic and does not cause apoptosis.

### REFERENCES

Cohen F E, Kelly J W. (2003) Therapeutic approaches to protein-misfolding diseases. Nature 426:905-909.
Gilchrist, C. A., Baker, R. T. (2000) Characterization of the ubiquitin-specific protease activity of the mouse/human Unp/Unph oncoprotein. Biochim Biophys Acta 1481, 297-309
Hobbs H H, Russell D W, Brown M S, Goldstein J L. (1990) The LDL receptor locus in familial hypercholesterolemia: mutational analysis of a membrane protein. Annu Rev Genet. 24:133-170.
Huang Y, Baker R T, Fischer-Vize J A. (1995) Control of cell fate by a deubiquitinating enzyme encoded by the fat facets gene. Science 270:1828-1831
Jensen T J, Loo M A, Pind S, Williams D B, Goldberg A L, Riordan J R. (1995) Multiple proteolytic systems, including the proteasome, contribute to CFTR processing. Cell 83:129-135.
Jörgensen M M, Jensen O N, Holst H U, Hansen J J, Corydon T J, Bross P, Bolund L, Gregersen N. (2000) Grp 78 is involved in retention of mutant low density lipoprotein receptor protein in the endoplasmic reticulum. J. Biol. Chem. 275:33861-33868.
Klinger, M., Kuhn, M., Just, H., Stefan, E., Palmer, T., Freissmuth, M., Nanoff, C. (2002) Removal of the carboxy terminus of the A_{2A}-adenosine receptor blunts constitute activity: Differential effect on cAMP accumulation and MAP kinase stimulation. Naunyn Schmiedeberg's Arch. Pharmacol. 366:287-298
Kostova Z, Wolf D H. (2003) For whom the bell tolls: protein quality control of the endoplasmic reticulum and the ubiquitin-proteasome connection. EMBO J. 22:2309-2317.
Kudlacek, O., Mitterauer, T., Nanoff, C., Hohenegger, M., Tang, W.-J., Freissmuth, M., and Kleuss, C. (2001) Inhibition of adenylyl and guanylyl cyclase isoforms by the antiviral drug foscarnet. J. Biol. Chem. 276:3010-3016
Kupershmidt S, Yang T, Chanthaphaychith S, Wang Z, Towbin J A, Roden D M. (2002) Defective human Ether-a-go-go related gene trafficking linked to an endoplasmic reticulum retention signal in the C terminus. J. Biol. Chem. 277:27442-27448.
Moazed, D., Johnson, D. (1996) A deubiquitinating enzyme interacts with SIR4 and regulates silencing in S. cerevisiae. Cell 86:667-677
Oksche A, Rosenthal W. (1998) The molecular basis of nephrogenic diabetes insipidus. J. Mol. Med. 76:326-337.
Palmer T M, Poucher S M, Jacobson K A, Stiles G L. (1995) ¹²⁵I-4-(2-[7-amino-2-[2-furyl][1,2,4]triazolo[2,3-a][1,3,5]triazin-5-yl-amino]ethyl)phenol, a high affinity antagonist radioligand selective for the A₂ₐ-adenosine receptor. Mol. Pharmacol. 48:970-974.
Pankevych H, Korkhov V, Freissmuth M, Nanoff C. (2003) Truncation of the A₁-adenosine receptor reveals distinct roles of the membrane-proximal carboxyl terminus in receptor folding and G protein coupling. J. Biol. Chem. 278:30283-30293
Papa F. R., Hochstrasser M. (1993) The yeast DOA4 gene encodes a deubiquitinating enzyme related to a product of the human tre-2 oncogene. Nature 366:313-319.
Pasyk E A, Foskett J K. (1995) Mutant (delta F508) cystic fibrosis transmembrane conductance regulator C1-channel is functional when retained in endoplasmic reticulum of mammalian cells. J. Biol. Chem. 270:12347-12350.
Petaja-Repo U E, Hogue M, Laperriere A, Walker P, Bouvier M. (2000) Export from the endoplasmic reticulum represents the limiting step in the maturation and cell surface expression of the human delta opioid receptor. J. Biol. Chem. 275:13727-13736.
Petaja-Repo U E, Hogue M, Laperriere A, Bhalla S, Walker P, Bouvier M. (2001) Newly synthesized human delta opioid receptors retained in the endoplasmic reticulum are retrotranslocated to the cytosol, deglycosylated, ubiquitinated, and degraded by the proteasome. J. Biol. Chem. 276:4416-4423.
Puente, X. S. et al., Human and mouse proteases: a comparative genomic approach; Nat. Rev. Genet. 4(7), 544-558 (2003)
Rowntree R K, Harris A. (2003) The phenotypic consequences of CFTR mutations. Ann Hum Genet. 67:471-485.
Strausberg, R. L., et al.; Generation and initial analysis of more than 15,000 full-length human and mouse cDNA sequences; Proc. Natl. Acad. Sci. U.S.A. 99(26), 16899-16903 (2002)
Wing, Simon; (2003) Deubiquitinating enzymes—the importance of driving in reverse along the ubiquitin-proteaseome pathway. IJBCB 35:590-605
Zhu Y, Pless M. Inhorn R, Mathey-Prevot B, D'Andrea A D. (1996) The murine DUB-1 gene is specifically induced by the betac subunit of interleukin 3 receptor. Mol Cell Biol. 16:4808-4817.

What is claimed is:

1. The use of Bortezomib and/or a pharmaceutically acceptable salt or ester thereof for the manufacture of a medicament for enhancing the expression of membrane proteins on the cell surface.

2. The use according to claim 1, characterized in that the medicament additionally comprises a compound stimulating deubiquitinating activity in a cell.

3. The use according to claim 2, characterized in that said additional compound is selected from the group consisting of
a deubiquitinating enzyme and
a nucleic acid sequence encoding a deubiquitinating enzyme.

4. The use according to claim 3, characterized in that the deubiquitinating enzyme is selected from the group consisting of ubiquitin carboxy-terminal hydrolases (UCH) and ubiquitin specific proteases (USP).

5. The use according to claim 4, characterized in that the deubiquitinating enzyme is USP-4.

6. The use according to any one of the foregoing claims for the manufacture of a medicament for enhancing the expression of a protein selected from the group consisting of CFTR (cystic fibrosis transmembrane conductance regulator), V2-vasopressin receptor, LDL-receptor and HERG-K+-channel.

7. The use according to any one of the foregoing claims for the manufacture of a medicament for the treatment of a disease or condition selected from the group consisting of cystic fibrosis, diabetes insipidus, hypercholesterinaemia and long QT-syndrome-2.

8. Pharmaceutical composition, comprising Bortezomib and/or a pharmaceutically acceptable salt or ester thereof and a therapeutically effective amount of a compound stimulating deubiquitinating activity in a cell.

9. Pharmaceutical composition according to claim 8, characterized in that the compound stimulating deubiquitinating activity is selected from the group consisting of
a deubiquitinating enzyme
a nucleic acid sequence encoding a deubiquitinating enzyme.

10. Pharmaceutical composition according to claim 9, wherein the deubiquitinating enzyme is selected from the group consisting of ubiquitin carboxy-terminal hydrolases (UCH) and ubiquitin specific proteases (USP).

11. Pharmaceutical composition according to claim 9 or 10, characterized in that the deubiquitinating enzyme is USP-4.

12. A method for enhancing the expression of membrane proteins on a cell surface, comprising the step of treating the cell with a pharmaceutically effective amount of Bortezomib and/or a pharmaceutically acceptable salt or ester thereof.

13. Method according to claim 12, comprising the additional step of contacting the cell with a compound stimulating the deubiquitinating activity.

14. Method according to claim 13, wherein said compound increases the amount of deubiquitinating enzymes in the cell.

15. Method according to claim 14, wherein a compound selected from the group consisting of
a deubiquitinating enzyme and
a nucleic acid sequence encoding a deubiquitinating enzyme is introduced into the cell.

16. Method according to claim 15, wherein said deubiquitinating enzyme is selected from the group consisting of ubiquitin carboxy-terminal hydrolases (UCH) and ubiquitin specific proteases (USP).

17. Method according to claim 16, wherein the deubiquitinating enzyme is USP-4.

18. Method according to any one of claims 12 to 17, for enhancing the expression of a protein selected from the group consisting of CFTR (cystic fibrosis transmembrane conductance regulator), V₂-vasopressin receptor, LDL-receptor and HERG-K⁺-channel.

19. A method for treating a disease or condition selected from the group consisting of cystic fibrosis, diabetes insipidus, hypercholesterinaemia and long QT-syndrome-2, comprising the step of administering to a patient in need thereof a pharmaceutically effective amount of Bortezomib and/or a pharmaceutically effective salt or ester thereof.

20. Method according to claim 19, comprising the step of additionally administering to said patient a compound selected from the group consisting of
a deubiquitinating enzyme and
a nucleic acid sequence encoding a deubiquitinating enzyme.
